(19) United States
(12) Patent Application Publication
(10) Pub. No.: US 2026/0174703 A1
(43) Pub. Date: Jun. 25, 2026

(54) A GELATIN COMPOSITION AND ITS USE IN THE PRODUCTION OF GELATINE CAPSULES

(71) Applicant: Tessenderlo Group NV, Brussels (BE)

(72) Inventors: Claudio Jose de Oliveira JACOMASSI, Brussels (BE); Vagner Barbosa SENA, Brussels (BE); Faye MAERTENS, Brussels (BE)

(21) Appl. No.: 19/123,803

(22) PCT Filed: Oct. 26, 2023
(86) PCT No.: PCT/EP2023/079993

§ 371 Date: Apr. 24, 2025

(30) Foreign Application Priority Data
Oct. 27, 2022 (EP) 22204254.1

(51) Int. Cl. A61K 9/48; A61K 47/26

(52) U.S. Cl. CPC A61K 9/4816; A61K 9/4825; A61K 9/4833; A61K 47/26

(57) ABSTRACT

The present invention relates to the field of gelatin capsules and in particular to the provision of a gelatin composition for use in the manufacture of gelatin capsules, in particular soft gelatin capsules. The composition of the present invention is particularly useful to reduce cross linking over time in capsules, improving capsule stability.

### TECHNICAL FIELD

The present invention relates to the field of gelatin capsules and in particular to the provision of a gelatin composition for use in the manufacture of gelatin capsules, in particular soft gelatin capsules. The composition of the present invention is particularly useful to provide stable soft capsules by reducing cross linking in said capsules.

### BACKGROUND

Capsules are widely used dosage forms in pharmaceutical and nutritional applications. Capsules comprise an outer part, the capsule shell, which is enclosing an inner part, the capsule filling. The capsule shell is typically manufactured with gelatin to which a certain amount of plasticizer and water is added. Two types of capsules exist on the market, hard capsules (i.e., hard-shell capsules) and soft capsules (i.e., soft-shell capsules). The shell of hard capsules is typically made of two parts, the cap and the body, while the shell of soft capsules is typically made of a single part. Hard and soft capsules are well-known in the art and are also called hard gelatin capsules and soft gelatin capsules.

Capsules are suitable to provide a certain dosage of a pharmaceutical composition or a nutritional supplement composition. The filling of the capsule can be solid (a powder, granulates), semi-solid (a paste) or liquid (an oily or aqueous solution or suspension).

When ingested, the gelatin of the capsule shell dissolves, allowing the capsule to release its filling which becomes available to be taken up by the body. The good performance of the capsule depends on the dissolution of its shell. With certain fillings however, and in particular in soft capsules, proper dissolution of the capsule shell can be reduced over the lifetime of the capsule, the storage stability of the capsules is thus reduced. One of the main reasons for this reduced dissolution over time is due to a cross linking reaction. Cross-linking can take place between the free functional groups of the gelatin of the capsule shell and certain components of the capsule filling; cross linking can also take place between gelatin molecules of the capsule shell. In both cases, the gelatin molecules become less prone to dissolution and as a result, the capsule shell needs more time to dissolve, or dissolves only partly and in extreme cases does not dissolve at all. This is unacceptable in particular when a pharmaceutical composition needs to be delivered to a patient.

The production process for soft gelatin capsules is well known. A gel mass is formed from a gelatin composition, which gel mass is then formed into a so-called gelatin ribbon or film. Two gelatin ribbons will get sealed with the help of die rolls and formed into a capsule shell. Simultaneously as the ribbons are sealed, a filling can be encapsulated and filled capsules are produced. The capsules are then dried. For efficient and smooth manufacturing, the gel mass should be easily formed, and have the right viscosity and stickiness for ribbon formation. During the drying step, it is also crucial to have the right rheological properties. Too much stickiness of the capsule shells will pose problems during drying.

CN103800912A provides a stable soft capsule shell containing 20-32 wt % hydrolyzed protein, 24-36 wt % gelatin, 5-9 wt % water, 17-27 wt % plasticizer, 7-18 wt % of disintegrating agent and 0.2-2.5 wt % of antioxidant. Hydrolyzed protein can be hydrolyzed soy protein, hydrolyzed rice protein, hydrolyzed wheat protein, hydrolyzed collagen, or a mixture thereof, more preferably hydrolyzed soy protein is used. However, soy protein hydrolysate is not very soluble, making gel mass formation difficult. Additionally, soy can be an allergen. Further, the use of soy hydrolysate negatively impacts the colour and turbidity of the capsule, resulting in opaque capsules. Regarding collagen hydrolysate, the present inventors have found that the use of such high amounts of collagen hydrolysate in combination with such low amounts of gelatin in the production of a capsule can be quite problematic due to stickiness of the gel mass and the gelatin ribbons made out of such gel mass, which also may result in improper sealing, and may result in leaking of the capsule filling. Also, drying may be negatively impacted dur to a too high stickiness of capsule shells. Furthermore, the use of high amount of disintegrating agent results in opaque capsules, which is less attractive to consumers.

U.S. Pat. No. 7,485,323B2 provides a gelatin composition comprising 5 to 10 wt % of a low molecular weight gelatine hydrolysate having a high primary amine content and 90 to 95 wt % of a gelatin, wherein the composition has an average viscosity of from about 15 to 50 cP.

US2020377924A1 relates to a process for the preparation of a low cross-linking gelatin blend obtained by selecting specific gelatin extracts during the gelatin production process.

The gelatin composition of the present invention aims at solving one or more of the above-mentioned problems, such as reducing cross linking, allowing the production of gelatin capsules with improved dissolution and improved storage stability, improving the production process of gelatin capsules, allowing the production of capsules which are properly sealed and have a reduced risk of leaking.

### SUMMARY OF THE INVENTION

The present invention relates to a gelatin composition comprising
a. from 50 wt % to 80 wt % (by weight of the gelatin composition) of gelatin and
b. from 20 wt % to 50 wt % (by weight of the gelatin composition) of collagen hydrolysate.

The gelatin composition of the invention shows improved rheological properties such as reduced stickiness upon formation of a gel mass, which simplifies the formation and production (including drying) of gelatin capsules. Further, the gelatin composition of the invention is useful to improve the shelf-life of soft capsules after production by reducing cross linking of gelatin from the capsule shell, thereby maintaining good dissolution of the capsule shell over time.

In a preferred embodiment, the gelatin composition is produced by co-drying of gelatin and collagen hydrolysate. Co-dried gelatin and collagen hydrolysate has improved powder properties and dissolves more easily, such that the gel mass is more easily formed.

The present invention further relates to the use of the gelatin composition of the present invention in the manufacture of gelatin capsules, preferably soft gelatin capsules.

The present invention further relates to a soft capsule shell comprising
a. From 37 wt % to 62 wt % (by weight of the capsule shell) of gelatin,
b. From 12 wt % to 31 wt % (by weight of the capsule shell) of collagen hydrolysate,
c. From 17 wt % to 30 wt % (by weight of the capsule shell) of plasticizer, and having a moisture content of from 5 wt % to 10 wt % (by weight of the capsule shell).

In a further aspect, the present invention relates to a method for producing a composition of co-dried gelatin and collagen hydrolysate.

### DETAILED DESCRIPTION OF THE INVENTION

The present invention relates to a gelatin composition comprising
a. From 50 wt % to 80 wt % (by weight of the gelatin composition) of gelatin and
b. From 20 wt % to 50 wt % (by weight of the gelatin composition) of collagen hydrolysate.

Preferably the gelatin composition of the present invention comprises, by total weight of the gelatin composition, from 51 wt % to 80 wt %, from 52 wt % to 80 wt, from 53 wt % to 80 wt %, from 54 wt % to 80 wt %, from 55 wt % to 80 wt %, from 56 wt % to 80 wt %, from 57 wt % to 80 wt %, from 58 wt % to 80 wt %, from 59 wt % to 80 wt %, from 60 wt % to 80 wt %, from 61 wt % to 80 wt %, from 62 wt % to 80 wt %, from 63 wt % to 80 wt %, from 64 wt % to 80 wt %, from 65 wt % to 80 wt % of gelatin.

Preferably further, the gelatin composition of the present invention comprises, by total weight of the gelatin composition, from 20 wt % to 49 wt %, from 20 wt % to 48 wt %, from 20 wt % to 47 wt %, from 20 wt % to 46 wt %, from 20 wt % to 45 wt %, from 20 wt % to 44 wt %, from 20 wt % to 43 wt %, from 20 wt % to 42 wt %, from 20 wt % to 41 wt %, from 20 wt % to 40 wt %, from 20 wt % to 39 wt %, from 20 wt % to 38 wt %, from 20 wt % to 37 wt %, from 20 wt % to 36 wt %, from 20 wt % to 35 wt % of collagen hydrolysate.

In a preferred embodiment, the gelatin composition of the present invention comprises from 60 wt % to 80 wt % (by weight of the gelatin composition) of gelatin and from 20 wt % to 40 wt % (by weight of the gelatin composition) of collagen hydrolysate.

In another preferred embodiment, the gelatin composition of the present invention comprises from 65 wt % to 80 wt % (by weight of the gelatin composition) of gelatin and from 20 wt % to 35 wt % (by weight of the gelatin composition) of collagen hydrolysate.

Preferably the composition has a moisture content of from 5 to 15 wt %, preferably of from 8 to 12 wt %, more preferably of from 5 to 10 wt %.

The composition may comprise one or more additives. The additive may be any type of suitable additive such as a vitamin, a mineral, a plasticizer, a colouring agent, stabilizer, flavouring, and the like.

Collagen is the main component of connective tissues in animals and humans. Collagen consists of chains of amino acids wound together to form triple-helices which in turn form elongated fibrils. It is mostly found in fibrous tissues such as tendons, ligaments, and skin. It is also found in bones, teeth, corneas, cartilage intervertebral discs and blood vessels. Collagen for industrial processing is mostly derived from animal skins and/or bones. Collagen may be processed to produce gelatin, which is obtained by irreversible, partial hydrolysis of collagen. According to the Food Chemical Codes, gelatin is defined as the product obtained from the acid, alkaline, or enzymatic hydrolysis of collagen, the chief protein component of the skin, bones, and connective tissues of animals.

A typical gelatin production process comprises several main steps to produce gelatin from raw materials:

Pre-treatment step: before processing, the raw materials are cleaned to remove foreign materials such as dirt, metal pieces etc.

Acid, alkaline, or enzymatic treatment of the raw materials results in the hydrolysis of collagen.

Extraction step: the raw materials are treated with warm water, during this step gelatin is extracted from the raw material matrix and solubilized in the water medium.

After the extraction step, gelatin is recovered by further steps of purification, concentration and drying.

In a collagen hydrolysate production process, the gelatin after the extraction step is further hydrolysed to obtain collagen hydrolysate which is then recovered by further steps such as purification, sterilisation, concentration and drying.

Gelatin is typically characterized by a gel strength (Bloom) and in warm solution by a certain viscosity. This gelatin can be further hydrolyzed to shorter protein chains to produce collagen hydrolysate or collagen peptides, losing its ability to form a gel, and becoming even soluble in water at ambient temperatures. Hydrolysis can be done by an enzymatic treatment with endoproteases or a combination of endo- and exoproteases for example. Chemical hydrolysis may also be done.

The gelatin can thus be obtained from a typical industrial process, from collagen containing material such as animal skin, preferably beef hide and/or pork skin and/or fish skin, more preferably from beef hide and/or pork skin; or such as animal bones, preferably from pig bones and/or beef bones and/or fish scales/bones and/or poultry bones, more preferably from pig bones and/or fish scales/bones.

Gelatin is further characterized by its viscosity. Said viscosity may be from 1.5 mPa·s to 7 mPa·s. Preferably, said viscosity is from 2.5 mPa·s to 5.5 mPa·s, more preferably from 4.0 mPa·s to 5.5 mPa·s, even more preferably from 4.5 mPa·s to 5.5 mPa·s. Viscosity is a well-known gelatin parameter in the art and is measured according to the method detailed below.

Gelatin is further characterized by its Bloom value. The Bloom value may range from 45 g Bloom to 325 g Bloom, preferably from 90 g Bloom to 300 g Bloom, more preferably from 100 g Bloom to 300 g Bloom, even more preferably of from 150 g Bloom to 300 g Bloom, yet even more preferably 200 g Bloom to 300 g Bloom. Bloom is a well-known gelatin parameter in the art and is measured according to the method detailed below.

Preferably the gelatin has an average molecular weight of from 20 kDa to 220 kDa, preferably from 30 kDa to 150 kDa, more preferably from 40 kDa to 120 kDa, even more preferably from 40 kDa to 110 kDa, yet even more preferably from 50 kDa to 100 kDa, yet even more preferably from 60 kDa to 90 kDa (average molecular weight (Mw) measured by gel permeation chromatography (Size exclusion chromatography using linear polymers such as polystyrene sulfonates or collagen chain fragments of different molecular weights as calibration vehicles). Average molecular weight is herein weight average molecular weight.

Preferably the collagen hydrolysate has an average molecular weight of from 300 to 5000 Da, preferably from 500 to 3000 Da, further preferably from 1000 to 3000 Da (average molecular weight (Mw) measured by gel permeation chromatography (Size exclusion chromatography using linear polymers such as polystyrene sulfonates or collagen chain fragments of different molecular weights as calibration vehicles). Average molecular weight is herein weight average molecular weight.

The composition of the present invention preferably has a transmittance at 450 nm (T %450 nm) of at least 50% and/or a transmittance at 620 nm (T %620) of at least 60%. More preferably, the transmittance at 450 nm is 60% or higher, even more preferably 65% or higher. More preferably, the transmittance at 620 nm is 70% or higher, even more preferably 75% or higher. A high transmittance indicates a low opacity of the final product, which is preferred by consumers.

Preferably the composition is a free-flowing composition, in the form of particulate solid. As used herein, the expression “particulate solid” is not particularly limited to the nature of the particulate solid and in particular includes granules, prills, pellets, pastilles, powders, strips, flakes. Preferably the composition is a powder.

Preferably, the composition of the present invention is characterized in that it has a substantially unimodal particle size distribution. A substantially unimodal particle size distribution as used herein means that the largest peak within the particle size distribution has an area at least 9 times as large as the next largest peak.

Such a substantially unimodal particle size distribution is advantageous in that the composition remains homogeneous during storage, transport, or manipulation of big bags. Such particle size distribution can be obtained for example when the collagen hydrolysate and the gelatin of the composition are co-dried, i.e., they are mixed together before the start of a drying step. After drying, the composition is grinded such as to obtain a particle size as defined above. Such a particle size distribution is also advantageous for use in a soft capsule manufacturing process. Indeed, such a particle size distribution is quite uniform and comprises few fine materials. The presence of such fine material is typically not desired by capsule manufacturers as it results in the production of foam, which eventually can cause capsule shells with air bubbles, improper sealing, and/or risk of leaking, if the foam formation is not properly controlled during the capsule production process, or if foam is not properly removed during the capsule production process.

Particle size distribution may be determined using mesh sizes. Such particle size may be measured at 8 mesh (2.36 mm opening), 10 mesh (2 mm opening), 14 mesh (1.40 mm opening), 20 mesh (0.85 mm opening), 40 mesh (0.425 mm opening), 60 mesh (0.250 mm opening), 100 mesh (0.150 mm opening), 200 mesh (0.075 mm opening), 400 mesh (0.038 mm opening). Preferably, at least 50 wt %, preferably at least 55 wt %, more preferably at least 60 wt %, even more preferably at least 65 wt %, yet even more preferably at least 70 wt %, yet even more preferably at least 75 wt %, yet even more preferably at least 80 wt %, yet even more preferably at least 85 wt %, yet even more preferably at least 90 wt % of the composition has the same particle size measured using the previous mesh sizes.

In one preferred embodiment, the composition of the present invention is provided in the form of a particulate solid having a particle size distribution wherein the main peak is measured at 14 mesh (1.40 mm opening). Preferably thus, at least 50 wt %, preferably at least 55 wt %, more preferably at least 60 wt %, even more preferably at least 65 wt %, yet even more preferably at least 70 wt %, yet even more preferably at least 75 wt % of the composition has a particle size of higher than 1.40 mm up to 2 mm. Further preferably at most 1 wt %, more preferably at most 0.5 wt % of the composition has a particle size of 0.425 mm or less.

In another preferred embodiment, the composition of the present invention is provided in the form of a particulate solid having a particle size distribution wherein the main peak is measured at 400 mesh (0.038 mm opening). Preferably thus, at least 50 wt %, preferably at least 55 wt %, more preferably at least 60 wt %, even more preferably at least 65 wt %, yet even more preferably at least 70 wt %, yet even more preferably at least 75 wt % of the composition has a particle size measured at 400 mesh, i.e., has a particle size of higher than 0.038 mm up to 0.075 mm. Further preferably at most 5 wt %, more preferably at most 3 wt %, more preferably at most 2 wt % of the composition has a particle size measured at 100 mesh, i.e., has a particle size higher than 0.150 mm. Further preferably at most 1 wt %, more preferably at most 0.5 wt % of the composition has a particle size measured at 60 mesh, i.e., has a particle size higher than 0.250 mm. Such particle size can be obtained by co-drying, alternatively, it can also be obtained by an agglomeration process, wherein at least part of the collagen hydrolysate is used as a binder.

The composition of the present invention can be produced by dry blending a desired amount of gelatin with a desired amount of collagen hydrolysate. Alternatively, in accordance with preferred embodiments of the invention, the composition of the present invention is thus produced by co-drying a desired amount of gelatin with a desired amount of collagen hydrolysate. Co-drying is advantageous in that the composition remains homogeneous during storage, transport, or manipulation of big bags. On the contrary, when the components are dry blended, a separation can occur during storage, manipulation, or transport, due to a difference in density and particle size distribution between the components. The separation can lead to irregular compositions, as collagen hydrolysate powder tends to have a smaller particle size and may be present in higher amounts at the top of the big bags, whereas the gelatin powder may be present at the bottom. It is then necessary to include a blending step before using the composition in a capsule manufacturing process for example. This additional blending step can be a disadvantage for the capsule manufacturer as this requires additional blending apparatus, storage tanks and ingredient manipulation.

Hence, in a further aspect of the invention there is provided a method for the production of a gelatin composition comprising the steps of:
(i) Providing an aqueous composition comprising collagen hydrolysate and gelatin, and
(ii) Drying said aqueous composition,
   wherein preferably the gelatin composition has
a. From 50 wt % to 80 wt % (by weight of the gelatin composition) of gelatin and
b. From 20 wt % to 50 wt % (by weight of the gelatin composition) of collagen hydrolysate.

Preferably a grinding step is performed after drying such as to obtain a composition having a desired particle size distribution. Alternatively, an agglomerating step is preformed after drying such as to obtain a composition having a desired particle size distribution.

In the method of the present invention, the gelatin composition is preferably the gelatin composition described herein. The preferred embodiments described herein for the gelatin composition (e.g., concentration and properties of the gelatin, concentration and properties of the collagen hydrolysate, particle size distribution, etc.) apply mutatis mutandis to the method for the production of a gelatin composition described herein.

Such a method is referred to herein as co-drying. Drying may be done by any suitable technique known in the art, such as spray drying, rotary drum drying, drying in a flow of hot air, belt drying, tunnel drying and the like. Drying is preferably performed using a tunnel dryer. Drying is performed until the composition has a moisture content of from 5 wt % to 15 wt %, preferably of from 8 wt % to 12 wt %, more preferably of from 5 wt % to 10 wt %.

The present invention preferably further relates to a gelatin composition obtainable by the co-drying method described herein. The gelatin composition obtainable by the co-drying method described herein is preferably the gelation composition described herein earlier, in particular in accordance with the preferred embodiments of the invention.

The aqueous composition of step (i) can be obtained in different ways.

Collagen hydrolysate and gelatin in powder form can be dissolved in water to provide the aqueous composition which is then dried.

Collagen hydrolysate can be produced in situ during the gelatin manufacturing process, after the gelatin extraction step. Gelatin is then dissolved in the extraction medium (typically water) and part of it can be further hydrolysed whereby an aqueous mixture of gelatin and collagen hydrolysate is produced. The mixture can then be dried in a suitable manner.

Preferably, the aqueous composition of step (i) is obtained during the manufacturing process of gelatin, wherein collagen hydrolysate, in powder or liquid form, is added to gelatin which is solubilized in the extraction medium after the gelatin extraction step. The mixture is then dried in a suitable manner.

Preferably thus, there is provided a method for the production of a gelatin composition comprising the steps of:
(i) Providing a raw material comprising collagen,
(ii) Treating the raw material comprising collagen with alkali, acid, or enzyme to hydrolyse collagen into gelatin,
(iii) Extracting gelatin in an aqueous medium at a temperature of from 40° C. to 90° C.,
(iv) Adding collagen hydrolysate and/or hydrolysing part of the gelatin to provide an aqueous composition comprising gelatin and collagen hydrolysate,
(v) Drying the composition obtained in step (iv),
(vi) Optionally grinding,
   wherein preferably the gelatin composition has
a. from 50 wt % to 80 wt % (by weight of the gelatin composition) of gelatin, and
b. from 20 wt % to 50 wt % (by weight of the gelatin composition) of collagen hydrolysate.

Alternatively, gelatin can be fully hydrolysed to produce collagen hydrolysate, to which gelatin (in powder or liquid form) is added before drying.

Preferably the aqueous composition of step (i) has a moisture content of from 20 wt % to 80 wt %, more preferably from 30 wt % to 70 wt %, even more preferably from 40 wt % to 60 wt %.

The present invention further relates to a soft capsule shell comprising the composition of the present invention and a plasticizer.

The present invention further relates to a soft capsule shell comprising:
a. from 37 wt % to 62% (by weight of the capsule shell) of gelatin,
b. from 12 wt % to 31 wt % (by weight of the capsule shell) of collagen hydrolysate,
c. from 17 wt % to 30 wt % (by weight of the capsule shell) of plasticizer, and having a moisture content of from 5 wt % to 10 wt % (by weight of the capsule shell).

Gelatin and collagen hydrolysate are as defined herein.

Preferably the capsule shell of the present invention comprises, by weight of the capsule shell, from 35 wt % to 55 wt %, more preferably from 37 wt % to 55 wt %, yet even more preferably 40 wt % to 55 wt %, yet even more preferably 40 wt % to 50 wt % of gelatin. In a further preferred embodiment, the capsule shell of the present invention comprises, by weight of the capsule shell, from 35 wt % to 60 wt %, more preferably from 37 wt % to 60 wt %, yet even more preferably 40 wt % to 60 wt % of gelatin. In a further preferred embodiment, the capsule shell of the present invention comprises from 35 wt % to 40 wt % of gelatin.

Preferably further, the capsule shell of the present invention comprises, by weight of the capsule shell, from 18 wt % to 30 wt %, more preferably from 18 wt % to 25 wt. %, even more preferably from 20 wt % to 25 wt % of collagen hydrolysate.

In a further preferred embodiment, the capsule shell of the present invention comprises, by weight of the capsule shell, from 20 to 31 wt % collagen hydrolysate, even more preferably between 25-31 wt % collagen hydrolysate, yet even more preferably between 25-30 wt % of collagen hydrolysate.

Preferably the capsule shell of the present invention comprises, by weight of the capsule shell, from 17 wt % to 37 wt %, more preferably 25 wt % to 37 wt %, even more preferably 27 wt % to 37 wt %, yet even more preferably 27 wt % to 35 wt % of a plasticizer. In a further embodiment, the capsule shell of the present invention comprises from 20 wt % to 35 wt %, more preferably from 20 wt % to 30 wt % of a plasticizer.

Suitable plasticizer may be glycerin, sorbitol, or propylene glycol, preferably the plasticizer is glycerine.

Preferably, the capsule shell of the present invention has a moisture content of from 5 wt % to 10 wt %, more preferably from 8 wt % to 10 wt %.

In a preferred embodiment, the capsule shell comprises from 40 wt % to 55 wt % (by weight of the capsule shell) of gelatin, from 18 wt % to 25 wt % (by weight of the capsule shell) of collagen hydrolysate, from 27 wt % to 35 wt % (by weight of the capsule shell) of plasticizer and from 5 wt % to 10 wt % (by weight of the capsule shell) of water. In a further preferred embodiment, the capsule shell of the present invention comprises, in addition to the preferred ranges described in this paragraph for gelatine, plasticizer and water, by weight of the capsule shell, from 20 to 31 wt % collagen hydrolysate, even more preferably between 25-31 wt % of collagen hydrolysate.

In a further preferred embodiment, the soft capsule shell of the invention comprises from 37 to 55 wt. % of gelatine, and/or from 20 to 31 wt. % collagen hydrolysate, more preferably 25-31 wt % collagen hydrolysate.

The combined weight percentages will not exceed 100%.

Preferably the capsule shell of the present invention comprises, by weight of the capsule shell, 10 wt % or less, preferably 7 wt % or less, more preferably 5 wt % or less, even more preferably 3 wt % or less of a disintegrant, more preferably it comprises substantially no disintegrant. A disintegrant may be any suitable disintegrant typically used in solid dosage forms such as capsules, tablets and the like and may be one or more of starch, sodium carboxymethyl starch or hydroxypropyl starch, maltodextrin and the like. Lower amounts of disintegrant allow increased transparency of the capsule.

Preferably further, the capsule shell of the present invention comprises, by weight of the capsule shell, less than 0.5 wt %, preferably less than 0.2 wt % of an antioxidant, preferably in that it contains substantially no antioxidant. An antioxidant may by any suitable antioxidant typically used in solid dosage forms such as capsules, tablets and the like and may be one or more of fumaric acid, cysteine, glycine, ethylenediaminetetraacetic acid, sodium hydrogen sulphite, sodium metabisulfite, sodium thiosulfate, vitamin C, and the like.

The capsule shell of the present invention is a soft capsule shell.

Preferably the capsule shell has a thickness of 0.60 to 1.5 mm, more preferably from 0.70 to 1 mm, even more preferably 0.75 to 0.95 mm.

Further, the present invention relates to a capsule, preferably a soft capsule, comprising the capsule shell of the present invention and a capsule filling.

The capsule filling can be any suitable filling for soft capsules such as a pharmaceutically active filling, a nutraceutical and the like. The filling can be a solid, a semi-solid, a paste, an emulsion, a liquid and the like.

It has been found that the capsule shell and the capsule of the present invention have a good dissolution and remain stable upon storage, i.e., have a good dissolution upon production and upon storage. The capsule shell and the capsule of the present invention can have a good dissolution upon storage of 1 year or more, preferably 18 months or more, more preferably 24 months or more after production. Storage conditions are normal storage conditions for capsules known in the art. The capsule shell and the capsule of the present invention are further characterized in that when put in 37° C. water under stirring, more than 70 wt. % is dissolved after 15 minutes. Preferably, more than 80 wt. % is dissolved after 15 minutes. After accelerated storage at 40° C. at 75% relative humidity, more than 70 wt. % of the capsule shell and the capsule of the present invention is dissolved after 15 minutes under stirring in 37° C. water. Preferably, more than 80 wt. % is dissolved after 15 minutes. The dissolution properties can also be seen on a film produced with the composition of the present invention. A film is similar to a capsule except that it is not formed into a capsule but has the shape of a sheet, it can be round, square, or having any other geometrical suitable from. A capsule and a film produced with a given gelatin composition will have similar dissolution properties. Thus, similar conclusions can be drawn between the dissolution properties of a film and the dissolution properties of a capsule produced with a given gelatin composition.

The capsule shell and capsule of the present invention may be produced according to methods known in the art such as plate process, rotary die process, reciprocating die process, accogel process, seamless process.

The present invention further relates to the use of the composition of the present invention in the production of capsule shells, more preferably soft capsule shells.

The present invention further relates to the use of the composition of the present invention in the production of capsules, more preferably soft capsules.

The present invention further relates to the use of the composition of the present invention as a capsule shell component, preferably as a soft capsule shell component.

The composition may be used as a blend or the gelatin and collagen of the present invention may be added separately during the production of a capsule shell or capsule, preferably the composition is used as a blend, as described herein above.

It has been found that the use of the composition of the present invention in the production of soft capsule shells and soft capsules is advantageous for the reduction of cross linking and thus to improve the storage stability of said capsule shell and capsule.

### Measurement Methods

Following measurement methods are used to determine product parameters outlined in this description.

Moisture content is measured by placing 5 g of the product in an oven at a temperature of 105° C. for 18 hours. The weight difference before and after drying is calculated as the moisture content.

The Bloom value is the mass in grams necessary to depress a standard plunger 4 mm into the gel having a concentration of 6.67% and matured at 10.0° C. for 17 h. A 6.67% solution of the gelatin sample is prepared in a wide-mouthed test bottle at 60° C., cooled to 10° C. and kept for 17 h for maturation at this temperature. The resulting gel is tested using a gelometer according to the GME Monograph Standardised Methods for the Testing of Edible Gelatine (version 12, May 2017).

The viscosity is measured according to British Standard at 6.66%, 60° C. and is expressed in mPa·s. It is measured as described in the GME Monograph Standardised Methods for the Testing of Edible Gelatine (version 12, May 2017).

Transmittance is defined as % T=(I/Iₒ)×100, wherein I is transmitted light and Iₒ is incident light. Transmittance of the composition is measured according to spectrophotometry, where a solution of 6.67 wt % of the composition at 60° C. is measured in a spectrophotometer at 450 nm and 620 nm.

### Dissolution

Capsule or film stability test is the measurement of the dissolution over time.

Dissolution test: a vessel is filled with 900 ml of water at 37° C., 50 rpm stirring. the capsule/film is put in a sinker at the bottom of the vessel at time 0.

At time t: (for instance 3 min, 9 min, 15 min and 45 min): 5 ml of liquid is removed from the vessel and transmittance is measured at 214 nm. Reference transmittance (0% dissolved) is measured on plain water, before the capsule is put in the vessel. The dissolution percentage at time t is calculated based on the transmittance measured at time t in comparison with the reference transmittance.

Particle size distribution is measured according to standard method ASTM E11:

Particle size is measured by passing the composition through a series of vibrating sieves put on top of each other, with the sieve at the bottom having the highest mesh size: 8 mesh (2.36 mm), 10 mesh (2 mm), 14 mesh (1.40 mm), 20 mesh (0.85 mm), 40 mesh (0,425 mm), 60 mesh (0,250 mm), 100 mesh (0,150 mm), 200 mesh (0,075 mm), 400 mesh (0,038 mm).

The present invention will be further illustrated by the non-limiting examples.

### EXAMPLES

### Example 1: Production of a Capsule According to the Present Invention

A gel mass according to Table 1 is prepared at 60° C. The gelatin is a type A or B gelatin and has an average molecular weight of 40 kDa to 100 kDa, a Bloom strength of 150 g Bloom to 300 g Bloom and a viscosity of 2.5 mPa·s to 5.5 mPa·s. The collagen hydrolysate has an average molecular weight of 1000 to 3000 Da. The ingredients of table 1 are mixed in a recipient which is put in a water bath having a temperature of 60° C. to form a gel mass.

The gel mass is then formed into capsules using a soft gelatin capsule manufacturing machine, no filling was used for this test. The capsules had a shell thickness of about 0.75 to 1 mm.

### As Comparative Experiment A, a Capsule According to Table 1 is Produced.

TABLE 1

| | Example 1 | Comparative experiment A | |
|---|---|---|---|
| Gelatin | 42 wt % | 63.6 | wt % |
| Collagen hydrolysate | 18 wt % | 0 | wt % |
| Plasticizer (Glycerin) | 30 wt % | 26.4 | wt % |
| Moisture | 10 wt % | 10 | wt % |

The dissolution is measured after capsule production (T0 in FIG. 1) according to the method described above (ref. method USP 711) for Example 1 and the Comparative Experiment A.

Capsules according to Example 1 and Comparative Experiment A are put in accelerated aging in a climatic chamber at 40° C., 75% relative humidity, for 6 months. After accelerated aging, the dissolution is measured (6M in FIG. 1).

In FIG. 1, the dissolution of the capsules over time is shown for Example 1 and for comparative experiment A, before (T0) and after accelerated aging (6M). It can be seen that the composition according to the invention showed improved dissolution, both before and after accelerated aging. Furthermore, where the comparative experiment showed a delay in dissolution after accelerated aging, this was not observed for the composition according to the invention. This shows that there was little to no cross-linking in the capsules according to the invention.

### Example 2: Production of a Film with the Composition According to the Invention

A gel mass according to Table 2 below is prepared at 60° C. The gelatin is a type A or B gelatin and has an average molecular weight of 40 kDa to 100 kDa, a Bloom strength of 150 g Bloom to 300 g Bloom and a viscosity of 2.5 mPa·s to 5.5 mPa·s. The collagen hydrolysate has an average molecular weight of 300 to 5000 Da. The ingredients of table 2 are mixed together in a recipient which is put in a water bath at 60° C. to produce the gel mass. The mixture is degassed, and a gelatin film is cast on glass plates to a thickness of about 0.75 mm to 1 mm. The film is put in a climatic chamber and stored under conditions of temperature and relative humidity until the film has a moisture content of 10 wt %.

As comparative experiment B, a film according to the below table 2 is produced.

TABLE 2

| | Example 2 | Comparative experiment B | |
|---|---|---|---|
| Gelatin | 42 wt % | 34.42 | wt % |
| Collagen hydrolysate | 18 wt % | 31.77 | wt % |
| Plasticizer (Glycerin) | 30 wt % | 24.81 | wt % |
| Moisture | 10 wt % | 9 | wt % |

Results of transmittance at 450 nm, 620 nm, setting time/temperature, G′ and melting time/temperature are provided below.

| | T450 | T620 | Setting | Setting | G′ | Melting | Melting |
|---|---|---|---|---|---|---|---|
| De- | nm | nm | time | temp | 22° C. | time | temp |
| scription | (%) | (%) | (sec) | (° C.) | (Pa) | (sec) | (° C.) |
| Example 2 | 86.2 | 96.5 | 112.9 | 28 | 84068 | 77.75 | 46.4 |
| Compar- | 80.1 | 94.3 | 125.2 | 24.5 | 51709 | 72.6 | 44.7 |
| ative | | | | | | | |
| example B | | | | | | | |

1. A gelatin composition characterized in that it comprises
a. from 50 wt % to 80 wt % of gelatin, based on the weight of the composition, and
b. from 20 wt % to 50 wt % of collagen hydrolysate, based on the weight of the composition.

2. The composition of claim 1, further characterized in that the collagen hydrolysate has an average molecular weight of from 300 to 5000 Da, preferably from 500 to 3000 Da, more preferably from 1000 to 3000 Da.

3. The composition of claim 1 or 2, further characterized in that the gelatin has an average molecular weight of from 40 kDa to 110 kDa.

4. The composition of any preceding claim, further characterized in that the gelatin has a Bloom strength of from 45 g Bloom to 325 g Bloom and/or a viscosity of from 2.5 mPa·s to 5.5 mPa·s.

5. The composition of any preceding claim, characterized in that it has a substantially unimodal particle size distribution.

6. The composition of any preceding claim, further characterized in that it has a transmittance at 450 nm of at least 50%, preferably 60% or higher, more preferably 65% or higher, and/or a transmittance at 620 nm of at least 60%, preferably 70% or higher, more preferably 75% or higher.

7. The composition of any preceding claim, for use in the production of gelatine capsules.

8. A soft capsule shell comprising
a. from 37 wt % to 62 wt % of gelatin, based on the weight of the capsule shell,
b. from 12 wt % to 31 wt % of collagen hydrolysate, based on the weight of the capsule shell,
c. from 17 wt % to 30 wt % plasticizer, based on the weight of the capsule shell, and
d. a moisture content of from 5 to 10 wt %.

9. The soft capsule shell of claim 8, comprising from 20 to 31 wt % of collagen hydrolysate.

10. The soft capsule shell of claim 8 or 9, further characterized in that it comprises from 37 to 55 wt. % of gelatin, and/or from 18 to 30 wt. % collagen hydrolysate.

11. A process to manufacture the composition of claim 5, comprising the steps of
(i) Providing an aqueous composition comprising collagen hydrolysate and gelatin,
(ii) Drying said aqueous composition, and
(iii) Optionally grinding or agglomerating, preferably grinding.

12. The process of claim 11, wherein the process comprises the steps of
(i) Providing a raw material comprising collagen,
(ii) Treating the raw material comprising collagen with alkali, acid, or enzyme to hydrolyse collagen into gelatin,
(iii) Extracting gelatin in an aqueous medium at a temperature of from 40° C. to 90° C.,
(iv) Adding collagen hydrolysate and/or hydrolysing part of the gelatin to provide an aqueous composition comprising gelatin and collagen hydrolysate,
(v) Drying the composition obtained in step (iv), and
(vi) Optionally grinding or agglomerating, preferably grinding.

13. The process of claim 11, wherein the aqueous composition of step (i) is obtained by dissolving gelatin and collagen hydrolysate in water.

14. Use of the composition of any of claims 1 to 7, in the production of soft gelatin capsule shells and soft gelatin capsules.

15. A capsule comprising a shell according to any one of claims 8 to 10 and a filling encapsulated by said shell.
